(19) United States
(12) Patent Application Publication
(10) Pub. No.: US 2007/0208182 A1
(43) Pub. Date: Sep. 6, 2007

(54) Zwitterionic Non-Linear Optophores And Devices Incorporating Same

(76) Inventor: Andrew John Kay, Wellington (NZ)

Correspondence Address: DANN, DORFMAN, HERRELL & SKILLMAN, 1601 MARKET STREET, SUITE 2400, PHILADELPHIA, PA, 19103-2307, US

(21) Appl. No.: 10/560,670

(22) PCT Filed: Jun. 17, 2004
(86) PCT No.: PCT/NZ04/00124

§ 371 Date: Jan. 25, 2007

(30) Foreign Application Priority Data
Jun. 18, 2003 (NZ) 526561

(51) Int. Cl. C07D 311/02

(52) U.S. Cl. 549/284

(57) ABSTRACT

The invention relates to zwitterionic second order non-linear optophores comprising a compound of general Formula I: wherein D is selected from: and L and R1-R5 are as herein defined. Also, provided are polymeric compositions incorporating these compounds. These optophores display a large and efficient non-linear optical response and therefore can be used in the production of optoelectronic devices.

### TECHNICAL FIELD

The present invention relates to a new class of second order non-linear optophores (optical chromophores) and processes for making them. The invention also relates to polymers containing the optical chromophores and devices incorporating the same.

### BACKGROUND OF THE INVENTION

In recent years there has been intense interest in molecules displaying large and efficient non-linear optical responses (μ.β) and in the electro-optic materials that permit the effective translation of these molecular properties into large, efficient and usable macroscopic responses. This interest is due to their potential application in emerging optoelectronic and photonic technologies.

Such materials contain molecules with highly polarisable electrons. The application of an electric field changes the electron polarisation, causing in an increase in the index of refraction. The resulting decrease in the velocity of light can be used to convert electric signals into optical signals.

In attempting to design all-plastic (i.e., all organic/polymeric) composite materials as active components of optoelectronic devices, a number of criteria need to be addressed. Ideally, the active molecules should display a large non-linear optical response while still retaining synthetic expediency, transparency at communications wavelengths, and compatibility either when doped or functionalised within a polymer matrix. The composite materials must also exhibit thermal and photostability and maintain the noncentrosymmetry of the poled optophore array (i.e. temporal stability of the EO effect).

As these properties are interrelated, the structural features of a molecule that optimise one property may attenuate another. Accordingly, there continues to be a need for suitable optophores with improved properties.

Generally, optophores comprise donor and acceptor moieties flanking a large, conjugated π-manifold which may contain auxiliary substituents to minimise potentially deleterious aggregation effects that occur when the compounds are constrained within a matrix.

Conventional optophores are based upon ‘left-hand-side’ (LHS) (Marder-Perry plot) systems in which the ground-state electron distributions are dominated by only modest levels of charge separation or ‘bond length alternation’. Such compounds are known to demonstrate positive solvatochromic behaviour.

For example, U.S. Pat. No. 6,067,186 describes compounds characterised as having tetherable N,N-dialkyanilino or N,N-dialkylaminothiopheno donor functionalities linked via a π-electron interconnect to heterocyclic systems that act as electron acceptors. Large pendant substituents may be placed on either or both of the interconnect or the electron acceptor heterocycle.

In contrast, optophores whose ground states are predominated by ‘zwitterionic’ electron distributions (‘right-hand-side’ (RHS) optophores) are expected to possess large ground-state dipole moments. This is considered to prevent efficient maximisation of macroscopic ‘material’ optical non-linearity as a result of unfavourable optophore dipole-dipole interactions during poling. As a result, those skilled in the art expect ‘right-hand-side’ optophores to be less useful in optoelectronic applications.

Surprisingly, it has been discovered that the zwitterionic non-linear optophores (NLOs) of the present invention achieve large molecular figures of merit (μ.β) and are suitable for construction into polymer compositions which have application in optoelectronic and photonic technologies.

Accordingly, it is an object of the present invention to provide optophores with improved physical properties which also display a large and efficient non-linear optical response, or at least to provide the public with a useful choice.

### SUMMARY OF THE INVENTION

The present invention relates to a new class of zwitterionic non-near optophores comprising a heteroaromatisable donor nucleus (D) connected to a cyanodicyanovinyldihydrofuran acceptor via a substituted polyenic linker (L), and synthesis of same.

In one aspect the invention provides a compound of the general Formula I: 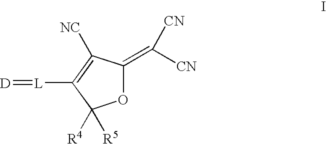
wherein
D is selected from the group comprising: 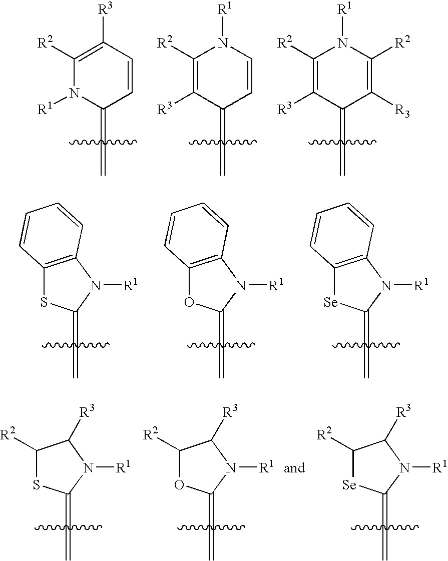
   and wherein:
   R¹ is alkyl or hydroxyalkyl;
   R² and R³ are H, or together with the carbon atoms to which they are attached form a 6-membered aromatic ring;
   L is a linker group comprising an optionally substituted chain of 3, 5 or 7 carbon atoms which, together with the double bond linking D to L forms a conjugated polyenic chain.
   R⁴ and R⁵ are independently alkyl hydroxyalkyl or p-C₆H₄—OAc.

In another the invention provides a compound of the general Formula I: 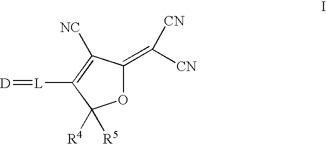
wherein:
D is selected from the group comprising: 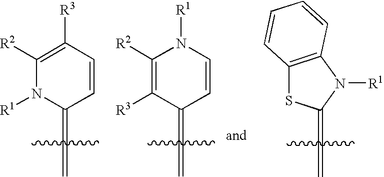
   and wherein:
   R¹ is alkyl or hydroxyalkyl;
   R² and R³ are H, or together with the carbon atoms to which they are attached form a 6-membered aromatic ring;
   L is a linker group comprising an optionally substituted chain of 3, 5 or 7 carbon atoms which, together with the double bond linking D to L forms a conjugated polyenic chain.
   R⁴ and R⁵ are independently alkyl, hydroxyalkyl or p-C₆H₄—OAc.

Preferably, L is a linker group comprising an optionally substituted chain of 3 or 5 carbon atoms which, together with the double bond linking D to L forms a conjugated polyenic chain.

Optionally, substituents may be added to the chain so as to minimise optophore dipole-dipole interactions and/or to rigidify the linker. These substituents may form cyclic structures with the p-electron backbone of the linker group.

Preferably, R¹ is monohydroxyalkyl or dihydroxyalkyl;

Preferably, R² and R³ together with the carbon atoms to which they are attached form a 6-membered aromatic ring;

Preferably, R⁴ and R⁵ are independently alkyl or hydroxyalkyl.

Particularly preferred are compounds of formula I as shown below: 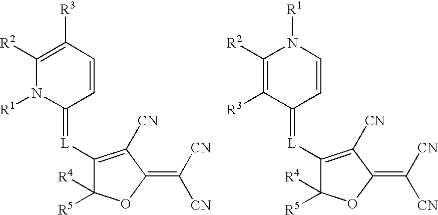
wherein:
R¹ is CH₃, CH₂CH₂OH, CH₂CH(OH)CH₂OH or alkyl chain of up to 30 carbon atoms;
R² and R³ are H or together with the carbon atoms to which they are attached form a 6-membered aromatic ring;
one of R⁴ or R¹ is hydroxyalkyl;
and L is an optionally substituted chain of 5 carbon atoms which, together with the double bond linking D to L forms a conjugated polyenic chain.

In particularly preferred compounds R¹ is dihydroxyalkyl. The provision of two hydroxyl groups allows the optophores of the invention to be used in the synthesis of new polyurethane, polycarbonate and polyamic acid/polyimide polymers.

The hydroxyalkyl group at R⁴ and/or R⁵ facilitates crosslinking of the polymers.

In another aspect the invention provides a method of preparing a compound of Formula I comprising:
(a) reacting a compound of Formula II: 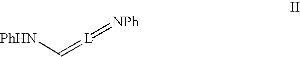
wherein L is defined as above, with a compound of Formula III: 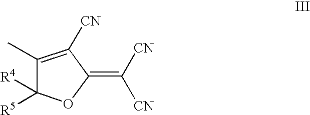
wherein R⁴ and R⁵ are as defined above, to form a compound of Formula IV: 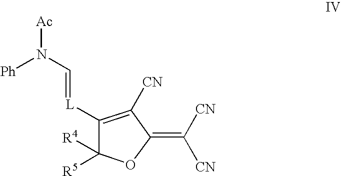
(b) reacting the compound of Formula IV from step (a) with a donor compound to form a compound of Formula I, wherein donor compound bears a donor group selected from the group comprising:

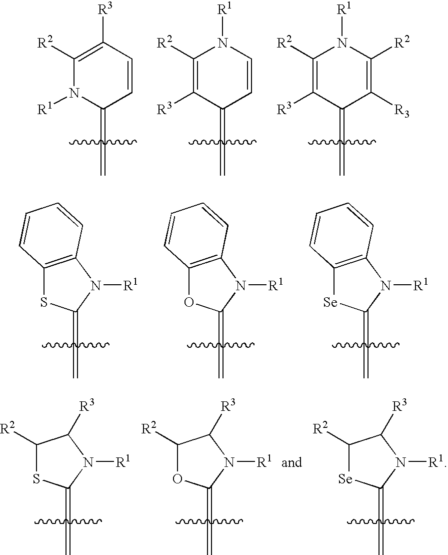

In another aspect the invention provides a method of preparing a compound of Formula I comprising:
(a) reacting a compound of Formula II: 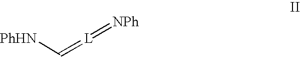
wherein L is defined as above, with a compound of Formula III: 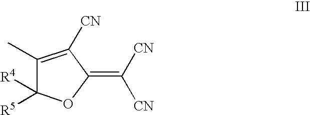
wherein R⁴ and R⁵ are as defined above, to form a compound of Formula IV: 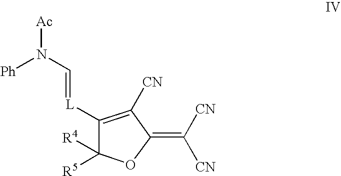
(b) reacting the compound of Formula IV from step (a) with an azinium or azolium donor derivative of Formula V, VI, or VII, where X is halogen, to form a compound of Formula I.

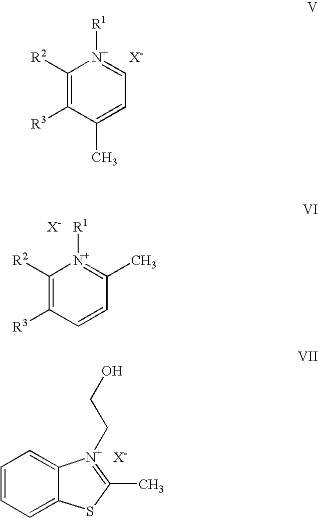

### DETAILED DESCRIPTION OF THE INVENTION

The term “alkyl” by itself or as part of another substituent, means a straight or branched chain or cyclic monovalent hydrocarbon radical which may be fully saturated, mono- or polyunsaturated.

The term “hydroxy” by itself or as part of another substituent, means an —OH group. Additionally, the term such “hydroxyalkyl” is intended to include polyhydroxyalkyl, for example, dihydroxyalkyl.

The term “aromatic ring” means an aromatic substituent which can be a single ring or multiple rings which are fused together covalently. The rings may contain from zero to four heteroatoms selected from N, O, and S, wherein the nitrogen and sulfur atom(s) are optionally oxidized, and the nitrogen atom(s) are optionally quaternized.

“Optoelectronic” pertains to having optical properties of a material alterable by an electric field.

A certain compound may exist in one or more particular geometric, optical, enantiomeric, diasteriomeric, epimeric, stereoisomeric, tautomeric, conformational, or anomeric forms, including but not limited to, cis- and trans-forms; E- and Z-forms; c-, t-, and r-forms; endo- and exo-forms; R-, S-, and meso-forms; D- and L-forms; (+) and (−) forms; keto-, enol-, and enolate-forms; α- and β-forms; axial and equatorial forms; boat-, chair-, twist-, envelope-, and halfchair-forms; and combinations thereof, hereinafter collectively referred to as “isomers” (or “isomeric forms”).

Note that, except as discussed below for tautomeric forms, specifically excluded from the term “isomers,” as used herein, are structural (or constitutional) isomers (i.e., isomers which differ in the connections between atoms rather than merely by the position of atoms in space). For example, a reference to a methoxy group, —OCH₃, is not to be construed as a reference to its structural isomer, a hydroxymethyl group, —CH₂OH. Similarly, a reference to ortho-chlorophenyl is not to be construed as a reference to its structural isomer, meta-chlorophenyl. However, a preference to a class of structures may well include structurally isomeric forms falling within that class (e.g., C₁₋₇alkyl includes n-propyl and iso-propyl; butyl includes n-, iso-, sec-, and tert-butyl; methoxyphenyl includes ortho-, meta-, and para-methoxyphenyl).

The above exclusion does not pertain to tautomeric forms, for example, keto-, enol-, and enolate-forms, as in, for example, the following tautomeric pairs: keto/enol (illustrated below), imine/enamine, amide/imino alcohol, amidine/amidine nitroso/oxime, thioketone/enethiol, N-nitroso/hyroxyazo, and nitro/aci-nitro.

Note that specifically included in the term “isomer” are compounds with one or more isotopic substitutions. For example, H may be in any isotopic form, including ¹H, ²H (D), and ³H(T); C may be in any isotopic form, including ¹²C, ¹³C, and ¹⁴C; O may be in any isotopic form, including ¹⁶O and ¹⁸O; and the like.

Unless otherwise specified, a reference to a particular compound includes all such isomeric forms, including racemic and other mixtures thereof. Methods for the preparation (e.g., asymmetric synthesis) and separation (e.g., fractional crystallisation and chromatographic means) of such isomeric forms are either known in the art or are readily obtained by adapting the methods taught herein in a known manner.

Unless otherwise specified, a reference to a particular compound also includes ionic, salt, hydrate, and protected forms of thereof, for example, as discussed below.

If the compound is cationic, or has a functional group which may be cationic (e.g., —NH may be —NH₃⁺), then a salt may be formed with a suitable anion. Examples of suitable inorganic anions include, but are not limited to, those derived from the following inorganic acids: hydrochloric, hydrobromic, hydroiodic, sulfuric, sulfurous, nitric, nitrous, phosphoric, and phosphorous. Examples of suitable organic anions include, but are not limited to, anions from the following organic acids: acetic, propionic, succinic, gycolic, stearic, lactic, malic, tartaric, citric, ascorbic, maleic, hydroxymaleic, phenylacetic, glutamic, benzoic, salicylic, sulfanilic, 2-acetyoxybenzoic, fumaric, toluenesulfonic, methanesulfonic, ethane disulfonic, oxalic, isethionic, and valeric.

It may be convenient or desirable to prepare, purify, and/or handle a corresponding solvate of the active compound. The term “solvate” is used herein in the conventional sense to refer to a

It may be convenient or desirable to prepare, purify, and/or handle a corresponding solvate of the active compound. The term “solvate” is used herein in the conventional sense to refer to a complex of solute (e.g., active compound, salt of active compound) and solvent. If the solvent is water, the solvate may be conveniently referred to as a hydrate, for example, a mono-hydrate, a di-hydrate, a tri-hydrate, etc.

It may be convenient or desirable to prepare, purify, and/or handle the active compound in a chemically protected form. The term “chemically protected form,” as used herein, pertains to a compound in which one or more reactive functional groups are protected from undesirable chemical reactions, that is, are in the form of a protected or protecting group (also known as masked or masking group). By protecting a reactive functional group, reactions involving other unprotected reactive functional groups can be performed, without affecting the protected group; the protecting group may be removed, usually in a subsequent step, without substantially affecting the remainder of the molecule. See, for example, Protective Groups in Organic J Synthesis (T. Green and P. Wuts, Wiley, 1991).

As referred to above, the invention provides a method for preparing a compound of Formula I. Each of the above mentioned steps is described in greater detail below.

In step (a), the cyanodicyanomethylidene dihydrofuran acceptor of Formula III is preferably reacted with an equimolar amount of a compound of Formula II. Preferably, step (a) is performed in acetic anhydride, however other solvents may be used such as methanol. An equivalent of sodium acetate may also be added when a bisanil hydrochloride salt is used.

The donor derivatives or donor compounds bearing a donor group (D) can be prepared using standard methods known in the art. Preferably, step (b) is performed by reacting stoichiometric quantities of each of the donor and acceptor components in refluxing acetic anhydride for 10 minutes. Preferably, the acetic anhydride contains an equivalent of triethylamine. Those skilled in the art will appreciate that other solvents and/or bases may be used in the method, and that the reaction time may differ depending on the nature of the reactants.

Substitution of the linker component can be achieved by modifying the bisanil component prior to reaction with the cyanodicyanovinyldihydrofuran acceptor of Formula III.

Substituents may form cyclic structures with the p-electron backbone of the linker group. For example, compounds wherein the linker comprises an alkylcycloalkenyl moiety (such as in Formula VIII) can be accessed using the chlorocyclohexene dialdehyde bisanil of Formula IX. Other linker components can be synthesised by nucleophilic substitution of the chlorine atoms in compounds such as VIII and IX by reaction with, for example, ROH, RSH or RNH₂ or by replacement with an alkyl substituent such as tert-butyl.

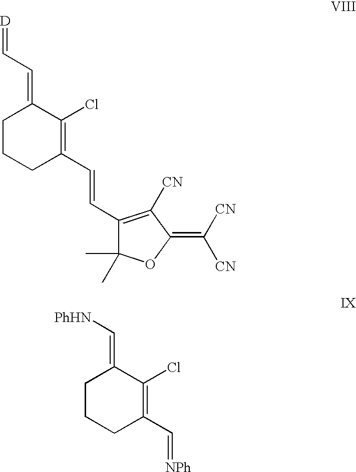

Other preferred substituents are thiophene and bithiophene pi-interconnects derived from bisanil precursors such as X and XI.

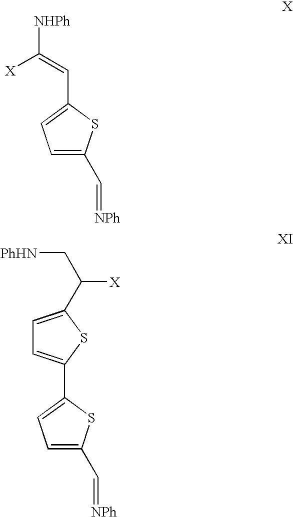

Using this methodology, compounds of Formula I incorporating a variety of different linker structures can be accessed by utilising the correct bisanil derivative. The method of the invention provides an expedient approach to the synthesis of a set of optophores because each different donor nuclei can be reacted with a pro-built (oligoen)amido substituted dihydrofuran acceptor group incorporating the actor and linker component.

It should be noted however, that the optophores of the invention can be synthesised using alternative methods. For example, the reaction of 4-[2-anilinovinyl]-1-(2-hydroxyethyl)pyridinium salt with cyanodicyanomethylidenedihydrofuran gives ‘{4{2-[N-(2-acetoxyethyl)pyridin-4(1H)-ylidene]ethenyl}-3-cyano-5,5-dimethyl-2(5H)furanylidene}’propanedinitrile (Formula I, R¹═CH₂CH₂OH, R², R³═H and R⁴, R⁵═CH₃, D=1,4-dihydropyridine).

Compounds displaying μ_{calc} β_{o(HRS)} values in excess of 15 000×10⁻⁴⁸ esu are considered to possess exceptional optical non-linearity. The optophores of the present invention give μ_{calc} β_{o(HRS)} values of up to 9384×10⁻⁴⁸ esu, and therefore show great potential for use in optoelectronic applications.

In a further aspect the invention provides a composite material prepared from a polymerization mixture comprising
(a) a compound of formula I or a derivative thereof; and
(b) at least one further polymerisable material

Preferably, the composite material comprises a modified polyurethane, polycarbonate, polyamic acid polyimide or a mixture thereof which includes substituents derived from a compound of formula I.

The composite material can be made using standard techniques known in the art.

For example an NLO polyurethane polymer can be made by reacting a compound of the invention wherein R¹ is 2,3-dihydroxypropyl with bisphenol-A and toluene-2,4 diisocyanate.

Employing this general methodology with varying quantities of the N-[2,3-dihydroxypropyl]-functionalised NLO chromophore, the sacrificial (non-NLO) dihydroxy component (above as bisphenol-A) and toluene-2,4 diisocyanate (or its equivalent), such that the sum of the mole ratios of the hydroxyl compounds is equal to that of toluene-2,4 diisocyanate (or its equivalent), enables the syntheses of many new NLO polyurethane polymers.

Furthermore, with the appropriate choice and use of tridentate hydroxyl-containing compounds (e.g triethanolamine) as components of the polymerisation process, it is possible to introduce measures of cross-linking/lattice-hardening to the NLO polymer material either prior to or during poling.

In like manner, it is possible to gain easy access to polymers belonging to the polycarbonate class of polymer. New polycarbonate polymers containing chromphores at variable loadings can be synthesised from appropriate mixtures of an NLO chromophore bearing the dihydroxpropyl tether (as above), a non-NLO (sacrificial) dihydroxy-containing component such as bisphenol-A and bisphenol-A chloroformate (or its equivalent).

In like manner, it is also possible to gain access to polymers belonging to the polyamic acid/polyimide classes of polymer. Functionalisation of each of the hydroxyl groups on the NLO chromphore, and on any sacrificial dihydroxy component (added so as to enable variation in chromphore loading) with trimellitic anhydride chloride, for example, and subsequent reaction of the resulting bisanhydrides with diamino substrates (aromatics, in particular) produces polyamic acids which can then be either chemically of thermally imidised.

Those skilled in the art will know that, by employing such polymer-tetherable NLO chromophores in conjunction with polymer-tetherable non-NLO active (sacrificial) spacer components, it is possible to gain access to innumerable condensation polymer systems with the appropriate choice of other reactant.

In addition, ready access can be gained to polymers that contain the NLO chromophore grafted to prescribed loadings in, for example, polymers that contain pendant carboxylic acid groups (by way of Mitsunobu coupling, for example).

In a yet further aspect the invention provides an optoelectronic device comprising the composite material of the invention.

The devices may include single elements or arrays of phase and amplitude optical modulators formed from the composite materials of the invention.

The functions of such devices include, but are not limited to: electrical to optical signal transduction; radio wave to millimeter wave electromagnetic radiation (signal) detection; radio wave to millimeter wave electromagnetic generation (broadcasting); optical and millimeter wave bean steering, and signal processing such as analog to digital conversion, ultrafast switching of signals at nodes of optical networks, and highly precise phase control of optical and millimeter wave signals.

The composite materials of the invention can be fabricated into a wide range of optoelectronic devices using standard protocols known in the art. Many articles and patents describe suitable techniques.

The invention also provides a method of data transmission comprising transmitting light through a composite material of the invention.

### EXAMPLES

The following examples are presented to further illustrate the practice of the invention.

### 4-[-2-Anilinovinyl]-1-(2-hydroxyethyl)pyridinium iodide

A mixture of N-(2-hydroxyethyl)pyridinium iodide (24 g) and N,N′-diphenylformamidine (18 g) was stirred at 120° C. for 1 h. On cooling the mixture formed a dark tar. This washed with 2×30 mL of ether and then allowed to stand, whereupon a brown-black solid formed. Recrystallisation of the solid from methanol afforded olive-green microcrystals (17.9 g; 54%), m.p. 192-193° C. (Found: C, 48.94; H, 4.69; N, 7.94. C₁₅H₁₇IN₂O requires C, 48.93; H, 4.65; N, 7.61%). ¹H NMR (d₆-DMSO) δ 10.30 (s, 1H), 8.50 (d, J 13.0 Hz, 1H), 8.33 (d, J 7.1 Hz, 2H), 7.77 (d, J 6.8 Hz, 2H), 7.33 (m, 4H), 7.03 (m, 1H), 5.89 (d, J 13.0 Hz, 1H), 5.13 (t, J 5.10 Hz, 1H), 4.30 (t, J 4.9 Hz, 2H), 3.78 (m, 2H). ¹³C NMR (d₆-DMSO) 155.4 (C_{Q}), 142.9 (CH), 142.6 (CH), 140.9 (C_{Q}), 129.9 (CH), 123.0 (CH), 119.0 (CH), 116.2 (CH), 99.1 (CH), 60.7 (CH₂), 60.4 (CH₂). μₘₐₓ (DMF) 428 log₁₀ε4.82.

### {3-Cyano-4,5-dimethyl-5-(4-hydroxyphenyl)-2(5H)-furanylidene}-propanedinitrie

Lithioethyl vinyl ether (^{t}BuLi/ethyl vinyl ether/THF/−78° C.) was reacted (−10° C.) with the TBDMS derivative of 4-hydroxyacetophenone, (5.0 g, 20.0 mmole) (M. He, T. M Leslie and J. A. Sinicropi, Chem. Mater., 2002, 14, 2393-4662; N. S. Wilson and B. A. Keay, Tetrahedron Lett., 1996, 37, 153). The crude intermediate α-ketol was then treated in solution in THF with 3 equivalents of tetrabutylammonium fluoride at ambient temperature for 2 hours after which time the solution was quenched with an ethyl acetate/water mixture. The organic phase was concentrated and subjected to flash chromatography over silica, eluting with 20% ethyl acetate/hexane, to give 3-hydroxy-3-(4-hydroxyphenyl)butan-2-one (2.3 g, 64%) as colourless needles, m.p. 101-102° C. (Found: C, 66.47; H, 6.79. C₁₀H₁₂O₃ requires C, 66.65; H, 6.71%). This deprotected ketol (5.0 g, 27.8 mmole) was then reacted with a mixture comprising malononitrile (9.1 g, 138.0 mmole), acetic acid (0.93 g, 15.5 mmole), and ammonium acetate (0.37 g, 4.8 mmole) in pyridine (45 ml) at ambient temperature for 16 hours. After this time, the red mixture was quenched into an ice/water slush and the resulting pink solid recovered by filtration. The purification of this solid was best accomplished by flash chromatography over silica eluting with 20-30% acetone/hexane, followed by recrystallisation from ethyl acetate/hexane after which the product was obtained (4.1 g, 51%) as a colourless crystalline solid, m.p. 225-227° C. (Found: C, 69.35; H, 3.89; N, 15.35. C₁₆H₁₁N₃O₂ requires C, 69.30; H, 4.00; N, 15.16%). ¹H NMR(CDCl₃) δ 9.37 (s, 1H), 7.03 (d, J 8.4 Hz, 2H), 6.90 (d, J 8.4 Hz, 2H), 2.21 (s, 3H), 1.98 (s, 3H).

### Synthesis of (N-acetyl-N-phenyl-oligoenamino)-2(5H)-furanylidene}propanedinitrile acceptors

General Condensation Procedure. (Step (a))

A mixture of the bisanil monohydrochloride (5.0 mmol), 4,5,5-trimethyl-3-cyano-2(5H)-furanylidenepropane dinitile (5.1 mmol) and anhydrous sodium acetate (5.1 mmol) in acetic anhydride was refluxed for 5-10 min before being allowed to cool and stand overnight. In the case of N,N′-diphenylformamidine free base, no sodium acetate was employed. Adducts were recovered by filtration as highly crane, coloured solids, and were washed with acetic anhydride (2×5 mL), followed by copious water and finally isopropanol. After drying in vacuum they were suitable for use without further purification.

{4-(2-Acetanilidoethenyl)-3-cyano-5,5-dimethyl-2(5H)-furanylidene}propanedinitrile Formula IV, L=(−) R⁴, R⁵═CH₃)

was purified by recrystallisation from acetone and isolated as yellow plates (79%), m.p. 274-278° C. (dec). (Found: C, 69.64, H, 4.57; N, 16.22. C₂₀H₁₆N₄O₂ requires C, 69.77; H, 4.65; N, 16.28%). Found: MH⁺ m/z 345.13460; C₂₀H₁₆N₄O₂ requires MH⁺ m/z 345.13480; Δ=0.6 ppm). ¹H NMR (d₆-DMSO) δ 8.72 (d, J 14.4 Hz, 1H), 7.63 (m, 3H), 7.47 (m, 2H), 5.10 (d, J 14.4 Hz, 1H), 2.05 (s, 3H), 1.66 (s, 6H). Irrespective of concentration, the ¹³C NMR spectrum was unexpectedly, but consistently, poor. Only the methyl and methine signals were identifiable. ¹³C NMR (d₆-DMSO)δ 145.1 (CH), 130.9 (CH), 130.5 (CH), 128.6 (CH), 98.7 (CH), 26.1 (CH₃), 23.5 (CH₃), λₘₐₓ (DMF) 430 log ε 4.74.

{4-(4-Acetanilido-trans-1,3-butadienyl)-3-cyano-5,5-dimethyl-2(5H)-furanylidene)-propanedinitrile (Formula IV, L=—C═C—, R⁴, R⁵═CH₃) was purified by recrystallisation from acetone and isolated as brick-red crystalline solid (86%), m.p. 272-274° C. (dec). (Found: C, 71.22; H, 4.58; N, 15.01. C₂₂H₁₈N₄O₂ requires C, 71.35; H, 4.86; N, 15.14%). Found: MH⁺ m/z 371.15025; C₂₂H₁₈N₄O₂ requires MH⁺ m/z 371.15141; Δ=3.1 ppm). ¹H NMR (d₆-DMSO) δ 8.54 (d, J 13.3 Hz, 1H), 7.91 (dd, J 15.3, 11.2 Hz, 1H), 7.57 (m, 3H, aromatic), 7.40 (m, 2H, aromatic), 6.40 (d, J 15.4 Hz, 1H), 5.41 (dd, J 13.3, 11.2 Hz, 1H), 1.99 (s, 3H), 1.70 (s, 6H). ¹³C NMR (d₆-DMSO) 177.5 (C_{Q}), 176.3 (C_{Q}), 169.7 (C_{Q}), 151.4 (CH), 145.3 (CH), 138.3 (CH), 130.6 (CH), 129.8 (CH), 128.8 (CH), 115.2 (CH), 113.4 (C_{Q}), 113.0 (CH), 112.6 (C_{Q}), 111.4 (C_{Q}), 98.8 (C_{Q}), 95.2 (C_{Q}), 52.6 (C_{Q}), 25.9 (CH₃), 23.5 (CH₃). λₘₐₓ (DMF) 526 log₁₀ε5.00.

{4-(6-Acetanilido-trans,trans-1,3,5-hexatrienyl)-3-cyano-5,5-dimethyl-2(5H)-furanyl-idene}-propanedinitrile (Formula IV, L=—C═C—C═C—, R⁴, R⁵ ═CH₃) was purified by recrystallisation from acetic anhydride as a purple crystalline solid (53%), m.p. 259-260° C. (Found: MH⁺ m/z 397.16590; C₂₄H₂₀N₄O₂ requires MH⁺ m/z 397.16535; Δ=1.4 ppm). ¹H NMR (d₆-DMSO) δ 8.04 (d, J 13.8 Hz, 1H), 7.70-7.47 (m, 4H), 7.60 (m, 1H), 7.37 (d, J 6.9 Hz, 2H), 7.30 (dd, J 14.4, 11.7 Hz, 1H), 6.45 (dd, J 14.1, 11.4 Hz, 1H), 6.42 (d, J 15.3 Hz, 1H), 5.18 (dd, J 13.8, 11.1 Hz, 1H), 1.92 (s, 3H), 1.69 (s, 6H). ¹³C NMR (d₆-DMSO) 177.4 (C_{Q}), 175.4(C_{Q}), 169.2(C_{Q}), 150.3(CH), 148.2(CH), 139.8(CH), 138.7(C_{Q}), 130.6 (CH), 129.6 (CH), 128.9 (CH), 116.1 (CH), 113.4(C_{Q}), 112.9, 112.6(C_{Q}), 111.6(C_{Q}), 98.8(C_{Q}), 96.2(C_{Q}), 53.5(C_{Q}), 25.8 (CH₃), 23.4 (CH₃). λₘₐₓ (DMF) 628 log₁₀ε5.02.

{4-[(3-Acetanilidomethylene)-2-chloro-1-cyclohexen-1-yl]-(E)-ethenyl-3-cyano-5,5-dimethyl-2(5H)-furanylidene}propanedinitrile (Formula IV, 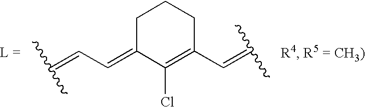
was recovered from the reaction mixture, washed with both acetic anhydride and then isopropanol and then dried to give the title compound as a purple crystalline solid (71%), m.p. 225-227° C. (Found: MH⁺ m/z 471.15925; C₂₇H₂₃N₄O₂ Cl requires MH⁺ m/z 471.15823; Δ=2.1 ppm). ¹H NMR (d₆-DMSO) δ 8.42 (d, J 16.0 Hz, 1H), 7.91 (s, 1H), 7.55-7.43 (m, 5H), 6.68 (d, J 16.0 Hz, 1H), 2.53, (m, 2H), 1.90 (1, 2H), 1.83 (s, 6H), 1.60 (m, 2H). ¹³C NMR (d₆-DMSO) 144.8 (CH), 134.8 (CH), 130.8 (C_{Q}), 129.5 (CH), 128.9 (CH), 117.1 (CH), 99.8 (C_{Q}), 27.5 (CH₂), 26.3 (CH₃), 21.9 (CH₂). No other lines were visible. λₘₐₓ (DMF) 502 log₁₀ε 4.64.

{4-(2-Acetanilidoethenyl)-5-(4-acetoxyphenyl)-3-cyano-5-methyl-2(5H)-furanylidene}-propanedinitrile (Formula IV, L=(−), R⁴═CH₃, R⁵=p-AcO—C₆H₄) was recovered by flash chromatography over silica (40% ethyl acetate/hexanes) and recrystallised from acetone/hexane to give yellow prisms (xx %), m.p. 253-256° C. (decomp.). ¹H NMR (d₆-acetone) δ 8.44 (d, J 14.2 Hz,1H), 7.67-7.60 (m, 5H), 7.43-7.41 (m,2H), 7.28 (d J 8.7 Hz, 2H), 5.29 (d, J 14.2 Hz, 1H), 2.30 (s, 3H), 2.23 (s,3H), 1.96 (s, 3H). ¹³C NMR (d₆-acetone) 147.0 (CH), 131.9 (CH), 129.6 (CH), 129.0 (CH), 124.0 (CH), 100.8 (CH), 25.1 (CH₃), 23.9 (CH₃), 21.5 (CH₃). No other lines were visible. λₘₐₓ (DMF) 434 log₁₀ε 4.78.

{4-[4-Acetamido-trans-1,3-butadienyl]-5-(4-acetoxyphenyl)-cyano-5-methyl-2(5H)-furanylidene}propanedinitrle (Formula IV, L=—C═C—, R⁴, ═CH₃, R⁵=p-AcO—C₆H₄) was synthesised using 4-hydroxyphenyl-substituted furanylidene propanedineitrile (Formula III, R¹═CH₃, R⁵═pH0-C₆H₄) and bisanil (Formula II, L=—C═), and recovered by flash chromatography over silica (30% acetone/hexanes) and isolated as maroon prisms (68%), m.p. 219-220° C. (Found: C, 71.17; H, 4.25; N, 11.66. C₂₉H₂₂N₄O₄ requires C; 71.02, H, 4.49; N, 11.43%. Found: MH⁺ m/z 491.17167; C₂₉H₂₂N₄O₄ requires MH⁺ m/z 491.17138; Δ=0.6 ppm). ¹H NMR (d₆-acetone) δ 8.15 (d, J 13.6 Hz, 1H), 7.63-7.20 (m, 9H), 7.48 (bd dd, 1H), 6.39 (d, J 15.3 Hz, 1H), 5.39 (dd, J 13.6, 11.4 Hz, 1H), 2.27 (s, 6H), 1.92 (s, 3H). ¹³C NMR (d₆-acetone) 178.0 (C_{Q}), 175.1 (C_{Q}), 170.1 (C_{Q}), 169.8(C_{Q}), 153.5 (C_{Q}), 151.9 (CH), 145.0 (CH), 139.7 (C_{Q}), 135.0 (C_{Q}), 131.7 (CH), 130.8 (CH), 129.8 (CH), 129.2 (CH), 123.8 (CH), 116.5 (CH), 113.5 (C_{Q}), 112.9 (C_{Q}), 111.9 (C_{Q}), 100.0 (C_{Q}), 24.5 (CH₃), 23.7 (CH₃), 21.4 (CH₃). λₘₐₓ (DMF) 530 log₁₀ε 4.99.

### Synthesis of II-Bridged Pyridinylidene and Quinolinylidene 2(5H)-Furanylidene}Propanedinitrile Optophores

General Condensation Procedures.

Method A: Equimolar quantities of the appropriate, the oligoenamido acceptor (Formula IV) and triethylamine were dissolved in acetic anhydride (10 ml/mmol) and the solution refluxed for 5-10 min before being allowed to cool slowly. Crystalline adducts were recovered by filtration and washed thoroughly with fresh acetic anhydride followed by copious quantities of water and then isopropanol and then dried. Yields were consistently in excess of 60%.

Method B: As for Method A, but with methanol as the solvent instead of acetic anhydride. Method C: Equimolar quantities of N-(2,3-dihydroxypropyl)-4-picolonium chloride (A. J. Kay, A. D. Woolhouse, G. J. Gainsford, T. G. Haskell, T. H. Barnes, L. T. McKinnie and C. P. Wyss, J. Mater. Chem., 2001, 11, 996.) and the oligoenamido acceptor (Formula IV) were treated with catalytic triethylamine in refluxing acetic anhydride as described above. The cooled reaction mixtures were poured into ether (ca. 25 ml/mmol reagent) and stirred vigorously for several minutes. The liquors were decanted and the oily residues washed by stirring with further portions of ether. The residual insoluble oils were stirred vigorously with aqueous sodium hydroxide solution (2% w/v, 25 ml/mmol) at 90° C. for 30 min and the resulting solids recovered by filtration and washed with water (to neutrality) followed by isopropanol and then dried. Yields of the optophores recovered in this manner were again in excess of 55%.

### (i) N-Methylpyridin-4(1H)-ylidene donors—Method A

[4{2-(N-Methylpyridin 4(1H)-ylidene)ethenyl}-3-cyano-5,5-dimethyl-2(5H)furanylidene}]-propanedinitrile (1a) was obtained as a grey/green microcrystalline solid (83%), m.p.>300° C. Found: MH⁺ m/z 317.13969; C₁₉H₁₇N₄O requires MH⁺ m/z 317.13910; Δ=1.8 ppm). ¹H NMR (d₆-DMSO) δ 8.48 (d, J 6.9 Hz, 1H), 8.41 (d, J 7.0 Hz, 1H), 8.35 (dd, J 14.8, 12.7 Hz, 0.5H), 7.86 (d, J 7.0 Hz, 1H), 7.63 (dd, J 14.4,12.7 Hz, 0.5H), 7.53 (d J 7.0 Hz, 1H), 6.34 (d J 14.5 Hz, 0.5H), 6.28 (d, J 14.8 Hz, 0.5H), 5.73 (d, J 12.5 Hz, 0.5H), 5.65 (d, J 12.1 Hz, 0.5H), 4.07 (s, 3H), 1.66 (s, 3H), 1.43 (s, 3H). ¹³C NMR (d₆-DMSO) 160.5 (C_{Q}), 159.4 (C_{Q}), 153.3 C_{Q}), 144.1 (CH), 143.6 (CH), 138.3 (CH), 138.2 (CH), 121.5 (CH), 120.7 (CH), 118.6 (CH), 117.6 (CH), 116.8 (C_{Q}), 115.4 (C_{Q}), 104.6 (CH), 103.9 (CH), 92.9 (C_{Q}), 92.6 (C_{Q}), 46.2 (CH₃), 46.1 (CH₃), 27.6 (CH₃), 27.4 (CH₃). λₘₐₓ (DMF) 570 log₁₀ε 4.86; (MeOH) 564; (pyridine) 600.

[4{4-(N-Methylpyridin-4(1H)-ylidene)-1,3-butadienyl}-3-cyano-5,5-dimethyl-2(5H)-furanylidene]propanedinitrile (1b) was obtained as a purple-blue powder (60%), 282-284° C. (Found: C, 73.12; H, 5.40, N, 16.25. C₂₁H₁₈N₄O requires C, 73.60; H, 5.20; N, 16.36%. Found: MH⁺ m/z 343.15534; C₂₁H₁₉N₄O requires MH⁺ m/z 343.15484 Δ=1.4 ppm). ¹H NMR (d₆-DMSO) δ 8.53 (d, J ca 6.9 Hz, 2H), 7.96 (d, J 6.9 Hz, 1H), 7.81 (d, J 6.7 Hz, 1H), 7.80 (t, J 13.2 Hz, 0.5H), 7.60 (m, 1H), 7.07 (t, J 13.2 Hz, 0.5H), 6.59 (d, J 15.3 Hz, 0.5H), 6.51 (d, J 15.3 Hz, 0.5H), 6.29 (m, 1H), 5.64 (d, J 12.9 Hz, 0.5H), 5.53 (d, J 12.3 Hz, 0.5H), 4.1 (s, 3H), 1.60 (s, 3H), 1.39 (s, 3H). ¹³C NMR (d₆-DMSO) 156.5 (C_{Q}), 154.3 (C_{Q}), 153.2 (C_{Q}), 153.1 (C_{Q}), 144.7(CH), 143.5(CH), 139.9 (CH), 139.0 (CH), 125.9 (CH), 125.1 (CH), 121.7 (CH), 121.5 (CH), 120.5 (CH), 118.1 (C_{Q}), 115.8 (C_{Q}), 105.2 (CH), 104.9 (CH), 92.2 (C_{Q}), 91.8 (C_{Q}), 46.4 (CH₃), 27.6 (CH₃), 27.3 (CH₃). λₘₐₓ (DMF) 600 log₁₀ε 4.78; (MeOH) 592; (pyridine) 670.

[4{6-(N-Methylpyridin-4(1H)-ylidene)-1,3,5-hexatrienyl}-3-cyano-5,5-dimethyl-2(5H)-furanylidene]propanedinitrile (1c) was obtained as a dark green powder (7%), m.p. 231-233° C. (Found: MH⁺ m/z 369.17099; C₂₃H₂₀N₄O requires MH⁺ m/z 369.16929=4.6 ppm). ¹H NMR (d₆-DMSO) δ 8.61 (bd t, J 6.4 Hz, 2H), 7.91 (bd t, J 6.2 Hz, 2H), 7.74 and 7.63 (2×dd J 15.2, 11.2 Hz and 15.0, 11.3 Hz respectively, approx 2:1 in intensity, 1H), 7.41 (bd t or dd, J 13.0 Hz, 0.67H), 6.96-6.73 (m, 1.5H), 6.59 (bd d, J 16.4 Hz, 1H), 6.42 (bd dd, J 14.10, 11.4 Hz,1H), 6.19 (bd ‘q’, J 13.4 Hz, 1H), 5.57 and 5.46 (2×d, J 12.6 and 12.1 Hz respectively, approx 1:2 in intensity, 1H), 4.1 (s, 3H), 1.56 and 1.37 (2×s, approx 1:2 in intensity, 6H). Solvent insolubility precluded the recording of meaningful ¹³C NMR data. λₘₐₓ (DMF) 615 log₁₀ε4.76; (MeOH) 600; (pyridine) 688.

### (ii) N-(2,3-Dihydroxypropyl)pyridin-4(1H)-ylidene donors—Method C

‘{4{2-[N-(2,3-Dihydroxypropyl)pyridine-4(1H)-ylidene]ethenyl}-3-cyano-5,5-dimethyl-2(5H)-furanylidene}’propanedinitrile (2a) was obtained as a blue grey powder (59%), m.p. 281-283° C. (Found: MH⁺ m/z 377.16082; C₂H₂₁N₄O₃ requires MH⁺ m/z 377.16085 Δ=0.1 ppm). ¹H NMR (d₆-DMSO), isomer 1, δ 8.79 (d, J 6.6 Hz, 1H), 8.37 (dd J 14.7, 12.0 Hz, 0.5H), 7.53 (d, J 6.6 Hz, 1H), 6.29 (d, J 14.7 Hz, 0.5H), 5.67 (d, J 12.0 Hz, 0.5H), 1.44 (s, 3H); isomer 2, δ 8.37 (d J 6.6 Hz, 1H), 7.86 (d J 6.6 Hz, 1H), 7.61 (dd, J 14.4, 12.9 Hz, 0.5H), 6.36 (d, J 14.4 Hz, 0.5H), 5.76 (d, J 12.3H, 0.5H), 1.67 (s, 3H). Resonances attributable to the dihydroxypropyl substituent were coincident for each isomer at δ 5.34 (dd, J 7.2,5.7 Hz, 1H, —CH₂O), 4.96 (t J 5.4 Hz, 1H, —CHOH), 4.48 (dd, J 13.6, 3.0 Hz, 1H, lower field branch of AB quartet, —NCH₂), 4.20 (m, 1H, higher field branch of AB quartet, —NCH₂), 3.83 (bd m, 1H, —CHOH), 3.48 (m, 1H, lower field branch of AB quartet, —CH₂OH), 3.33 (m, 1H, higher field branch of AB quartet, —CH₂OH). ¹³C NMR (d₆-DMSO, no quaternary resonances cited) 144.0 (py-CH), 143.5 (py-CH), 138.6 (CH), 138.5 (CH), 121.2 py-CH), 120.4 (py-CH), 118.5 (CH), 117.6 (CH), 104.6 (CH), 104.0 (CH), 70.8 (CH), 63.3 (CH₂), 62.1 (CH₂), 27.6(CH₃), 27.4(CH₃). λₘₐₓ (DMF) 572 log₁₀ε 4.79; (MeOH) 570; (pyridine) 598.

‘{4{4-[(2,3-Dihydroxypropyl)pyridin-4(1H)-ylidene]-1,3-butadienyl}-3-cyano-5,5-dimethyl-2(5H)-furanylidene}’propanedinitrle (2b) was obtained as a green powder powder (58%), m.p. 257-259° C. (Found: M{m/z 403.17832; C₂₃H₂₃N₄O₃ requires MH⁺ m/z 403.17789 Δ=3.5 ppm). ¹H NMR (d₆-DMSO) two isomers, approximately 1:1, δ 8.51 (bd d, c.7 Hz, 2H), 7.96 (d, J 6.9 Hz, 1H), 7.80 (d+m, J 6.9 Hz, 1.5H), 7.65 (m, 1H), 7.09 (t, J 13.2 Hz, 0.5H), 6.50 (2×d, J 14.8, 14.5 Hz, 1H), 6.35 (bd ‘q’. J 11.5 Hz, 1H), 5.66 (d, J 12.8 Hz, 0.5H), 5.53 (d, J 12.2 Hz, 0.5H), 5.35 (bd s, 1H, —OH), 4.96 (bd s 1H, —OH), 4.52 (bd d, J c, 13 Hz, 1H, lower field branch of AB quartet, —NCH₂), 4.22 (m, 1H, higher field branch of AB quartet, —NCH₂), 3.84 (bd ‘s’, 1H, —CHOH), 3.48 (m, 1H lower field branch of AB quartet, —CH₂OH), 3.33 (m, 1H, higher field branch of AB quartet, —CH₂OH), 1.60 (s, 3H), 1.40 (s, 3H). ¹³C NMR (d₆-DMSO, no quaternary resonances cited) 144.9 (CH), 144.1 (2py-CH), 143.7 (CH), 140.0 (CH), 139.2(CH), 126.0 (py-CH), 125.2 (py-CH), 122.0 (CH), 121.4 (CH), 120.5 (CH), 105.3 (CH), 105.0 (CH), 70.8 (CH), 63.3 (CH₂, 62.3 (CH₂) 27.6 (CH₃), 27.3 (CH₃). λₘₐₓ (DMF) 604 log₁₀ε 4.64; (MeOH) 598; (pyridine) 662.

### (iii) N-Methylpyridin-2(1H)-ylidene donors—Method A

[4{2-(N-Methylpyridin-2(1H)-ylidene)ethenyl}3-cyano-5,5-dimethyl-2(5H)-furanylidene]propanedinitrile (5a) was obtained as a dark pule powder, (96%). m.p. >300° C. (Found: MH⁺ m/z 317.13969; C₁₉H₁₇N₄O requires MH⁺ m/z 317.13808; Δ=5.1 ppm). ¹H NMR (d₆-DMSO) two isomers, approximately 1:1, isomer 1 δ 8.57 (d, J 6.0 Hz, 0.5H), 8.41 (d, J 7.9 Hz, 0.5H), 8.21 (t, J 7.8 Hz, 0.5H), 7.67 (dd, J 13.8, 12.8 Hz, 0.5H), 7.46 (t, J 7.8 Hz, 0.5H), 6.55 (d, J 14.1 Hz, 0.5H), 5.93 (d, J 12.5 Hz, 0.5H); isomer 2 δ 8.51 (d, J 6.0 Hz, 0.5H), 8.38 (dd, J 14.4,12.1 Hz, 0.5H), 8.07 (t, J 7.8 Hz, 0.5), 7.79 (d, J 8.3, 0.5H), 7.41 (t, J 6.9 Hz, 0.5H), 6.44 (d, J 14.4 Hz, 0.5H), 5.76 (d, J 12.1 Hz, 0.5H), 4.31/4.32 (2×s, 3H), 1.67/1.45 (2×s, 6H). ¹³C NMR (d₆-DMSO) 176.5 (C_{Q}), 174.8 (C_{Q}), 161.7 (C_{Q}), 160.4 (C_{Q}), 153.4 (C_{Q}), 153.2 (C_{Q}), 145.1(CH), 144.7 (CH), 142.6 (CH), 141.7 (CH), 140.1 (CH), 123.5 (CM, 122.0 (CH), 121.2 (CH), 120.9 (CH), 118.2 (C_{Q}), 117.6 (C_{Q}), 116.9 (C_{Q}), 116.6 (C_{Q}), 115.4 (C_{Q}), 110.9 (CH), 110.4 (CH), 104.7 (CH), 103.9 (CH), 93.0 (C_{Q}), 92.7 (C_{Q}), 75.2 (C_{Q}), 71.6 (C_{Q}), 45.3 (CH₃), 27.6 (CH₃), 27.4 (CH₃). λₘₐₓ (DMF) 556 log₁₀ε4.90; (MeOH) 550; (pyridine) 580.

[4{4-(N-Methylpyridin-2(1H)-ylidene)-1,3-butadienyl}-3-cyano-5,5-dimethyl-2(5H)-furanylidene}propanedinitrile (5b) was obtained as a blue-black powder, (76%), m.p. 291-294° C. (Found: MH⁺ m/z 343.15534; C₁₉H₁₇N₄O requires MH⁺ m/z 343.15654; Δ=3.5 ppm). ¹H (d₆-DMSO) two isomers, approximately 1:1, δ 8.62 (m, 1H), 8.44 (d, J 7.1 Hz, 0.5H), 8.20 (m, 1.5H), 7.88 (dd, J 14.7, 11.3 Hz, 0.5H), 7.75-7.48 (m, 2H), 7.16 (‘t’, J 13.3 Hz, 0.5H) 6.64 (‘t’, J 15.1 Hz, 1H), 6.36 (m, 1H), 5.65 (d, J 12.8 Hz, 0.5H), 5.53 (d, J 12.2 Hz; 0.5H), 4.13/4.12 (2×s, 3H), 1.61, 1.41 (2×s, 6H), ¹³C NMR (d₆-DMSO) 175.7 (C_{Q}), 173.9 (C_{Q}), 157.5 (C_{Q}), 155.2 (C_{Q}), 153.2 (C_{Q}), 152.1 (C_{Q}), 146.7 (CH), 145.5 (CH), 145.3 (CH), 142.7 (CH), 142.5 (CH), 141.1 (CH), 140.1 (CH), 125.7 (CH), 124.8 (CH), 123.9 (CH), 123.1 (CH), 122.5 (CH), 122.1 (CH), 118.0 (C_{Q}), 117.4 (C_{Q}), 115.7 (C_{Q}), 114.2 (CH), 113.1 (CH), 105.0 (CH), 104.7 (CH), 92.3 (C_{Q}), 92.0 (C_{Q}), 74.2(C_{Q}), 70.1 (C_{Q}), 45.5 (CH₃), 27.6 (CH₃), 27.3 (CH₃). λₘₐₓ (DMF) 588 log₁₀ε 4.81; (MeOH) 582; (pyridine) 662.

[4{6-(N-Methylpyridin-2(1H)-ylidene)-1,3,5-hexatrienyl}-3-cyano-5,5-dimethyl-2(5H)-furanylidene]propanedinitrile (5c) was obtained as a bronze-brown solid, (34%), m.p. 200-202° C. (Found: MH⁺ m/z 369.17099; C₂₃H₂₁N₄O requires MH⁺ m/z 369.17246; Δ=4.0 ppm). Solvent insolubility precluded the recording of meaningful ¹H and ¹³C NMR data although it was possible to discern the presence of predominantly two isomers (2:1) principally from the two doublets at δ 5.59 and 5.48, the two N—CH₃ singlets at δ 4.22 and 4.16 and the two gem-methyl singlets at δ 1.56 and 1.37. λₘₐₓ 598 (DMF) log₁₀ε 4.29.

### (iv) N-(2,3-dihydroxypropyl)pyridin-2(1H)-ylidene donors—Method C

‘{4[2-N-(2,3-Dihydroxypropyl)pyridin-2(1H)-ylidene]ethenyl}-3-cyano-5,5-dimethyl-2(5H)-furanylidene}’propanedinitrie (6a) was obtained as a dark green powder, (71%), m.p. 285-288° C. (Found: MH⁺ m/z 377.16005; C₂₁H₂₀N₄O₃ requires MH⁺ m/z 377.16082; Δ=2.0 ppm). ¹H NMR (d₆-DMSO) two isomers, approximately 1:1; δ 8.50-8.33 (m, 2H), 8.23 (t, J 7.9 Hz, 0.5H), 8.09 (t, J 7.8 Hz 0.5H), 7.83 (d, J 8.4 Hz, 0.5H), 7.66 (‘t’, J 13.3 Hz, 0.5H), 7.50 (t, J 6.9 Hz, 0.5H), 7.44 (t, J 6.8 Hz, 0.5H), 6.59 (d, J 14.1 Hz, 0.5H), 6.52 (d, J 14.4 Hz, 0.5H), 5.90 (d, J 12.5 Hz, 0.5H), 5.72 (d, J 12.1 Hz, 0.5H), 5.31 (m, 1H), 5.01 (m, 1H) 4.74 (bd t, J 10.9 Hz, lower field branch of AB quartet, 1H), 4.22 (m, higher field branch of AB quartet, 1H), 3.84 (bd m, 1H), 3.53 (m, lower field branch of AB quartet, 1H), 3.46 (m, higher field branch of AB quartet, 1H), 1.67 (s, 3H), 1.45 (s, 3H). ¹³C NMR (d₆-DMSO) 176.5 (C_{Q}), 174.8 (C_{Q}), 161.4 (C_{Q}), 160.1 (C_{Q}), 153.3 (C_{Q}), 152.8 (C_{Q}), 145.8, (CHI), 145.5 (CH), 142.8 (CH), 141.9 (CH), 140.2 (CH), 139.9 (CH), 123.9 (CH), 122.5 (CH), 121.0 (CH), 120.6 (CH), 118.3 (C_{Q}), 117.6 (C_{Q}), 116.7 (C_{Q}), 115.4 (C_{Q}), 110.8 (CH), 110.3 (CH), 104.7 (CH), 103.8 (CH), 92.9 (C_{Q}), 92.7, (C_{Q}), 75.0 (C_{Q}), 71.5 (C_{Q}), 69.6 (CH), 63.7 (CH₂), 60.0 (CH₂), 59.8 (CH₂), 27.7 (CH₃), 27.4 (CH₃). λₘₐₓ 556 (DMF) log₁₀ε 4.72; (MeOH) 552; pyridine) 578.

‘{4{4-N-(2,3-Dihydroxypropyl)pyridin-2(1H)-ylidene)-1,3-butadienyl}-3-cyano-5,5-dimethyl-2(5H)-furanylidene}’propanedinitrile (6b) was obtained as a dark green powder, (62%), m.p. 244-245° C. (Found: MH⁺ m/z 403.17785; C₂₃H₂₂N₄O₃ requires MH⁺ m/z 403.17647; Δ=3=3.4 ppm). ¹H NMR (d₆-DMSO) two isomers, approximately 1:1; δ 8.49 (m, 2H), 8.21 (m, 1.5H), 7.86 (dd, J 14.6, 11.3 Hz, 0.5H), 7.78-7.49 (m, 2.5H), 7.16 (t, J 13.3 Hz, 0.5H) 6.72 (dd, J 14.5, 11.4 Hz, 1H), 6.33 (m, 1H), 5.65 (d, J 12.8 Hz, 0.5H), 5.53 (d, J 12.2 Hz, 0.5H), 5.33 (m,1H), 5.04 (m,1H), 4.78 (m, lower field branch of AB quartet, 1H), 4.25 (m, higher field branch of AB quartet, 1H), 3.82 (m, 1H), 3.54 (m, lower field branch of AB quartet, 1H), 3.44 (m, higher field branch of AB quark, 1H) 1.61 (s, 3H), 1.40 (s,3H). Solvent insolubility precluded the recording of meaningful ¹³C NMR data). λₘₐₓ (DMF) 590 log₁₀ε 4.61; (MeOEH) 584; (pyridine) 652.

### (v) N-(2-Hydroxyethyl)quinolin-4(1H)-ylidene donors—Method B

‘{4{2-[N-(2-Hydroxyethyl)quinolin-4(1H)-ylidene]ethenyl}-3-cyano-5,5-dimethyl-2(5H)-furanylidene}’propanedinitrile (3a) was obtained as a green-brown powder, (89%), m.p. >300° C. (Found: MH⁺ m/z 397.16615; C₂₄H₂₀N₄O₂ requires MH⁺ m/z 397.16964 Δ=0.6 ppm). ¹H NMR (d₆-DMSO) two isomers, approximately 1:1 δ 8.77-8.45 (m, 2.5H), 8.34-7.70 (m, 4H), 7.48 (d, J 6.3 Hz, 0.5H), 7.32 (d, J 13.3 Hz, 0.5H), 7.21 (d, J 14.0 Hz, 0.5H), 6.16 (d, J 12.7 Hz, 0.5H), 5.94 (d, J 12.3 Hz, 0.5H), 5.10 (bd s, 1H), 4.77 (bd ‘s’, 2H), 3.85 (bd ‘s’, 2H), 1.74 (s, 3H), 1.50 (s, 3H). ¹³C NMR (d₆-DMSO) 175.3 (C_{Q}), 163.9 (C_{Q}), 162.6 (C_{Q}), 152.5 (C_{Q}), 152.4 (C_{Q}), 146.0 (CH), 145.1 (CH), 141.3 (CH), 138.5 (C_{Q}), 134.1 (CH), 134.0 (CH), 128.0 (CH), 127.7 (CH), 126.2 (CH), 125.9 (CH), 125.6 (C_{Q}), 119.0 (CH), 118.7 (CH), 117.9 (C_{Q}), 117.1 (C_{Q}), 116.3 (C_{Q}), 114.5 (CH), 113.6 (CH), 112.1 (CH), 110.9 (CH), 106.8 (CH), 105.8 (CH), 93.6 (C_{Q}), 59.3 (CH₂), 57.7 (CH₂), 57.4 (CH₂), 27.7 (CH₃), 27.2 (CH₃). λₘₐₓ (DMF) 660 log₁₀ε 5.00; (MeOH) 654; (pyridine) 682.

‘{4{4-[N-(2-Hydroxyethyl)quinolin-4(1H)-ylidene]-1,3-butadienyl}-3-cyano-5,5-dimethyl-2(5H)-furanylidene}’propanedinitrile (3b) was obtained as a dark-green powder, (82%), m.p. >300° C. (Found: MH⁺ m/z 423.17985; C₂₆H₂₂N₄O₂ requires MH⁺ m/z 423.18155 Δ=4.0 ppm). ¹H NMR (d₆-DMSO) two isomers, approximately 1:1 δ 8.74 (d, J 6.8 Hz, 1H), 8.67 (bd ‘s’, 1H), 8.45-7.69 (m, 5H), 7.59-7.22 (m,2H), 6.51 (dd, J 13.3,12.0 Hz,1H), 5.77 (d, J 12.6 Hz, 0.5H), 5.65 (d, J 12.1 Hz,0.5H), 5.13 (t, J 5.4 Hz,1H), 4.84 (bd s,2H), 3.86 (m,2H), 1.65 (bd s,3H), 1.44 (bd s, 3H). Solvent insolubility precluded the recording of meaningful ¹³C N data. λₘₐₓ (DMF) 735 log₁₀ε 4.88; (MeOH) 724; (pyridine) 782.

‘{4{6-[N-(2-Hydroxyethyl)quinolin-4(1H)-ylidene]-1,3,5-hexatrienyl}-3-cyano-5,5-dimethyl-2(5H)-furanylidene}’propanedinitrile (3c) was obtained as a green-black powder, (44%), m.p. 252-254° C. (Found: MH⁺ m/z 449.19545; C₂₈H₂₄N₄O₂ requires MH⁺ m/z 449.49720 Δ=3.9 ppm). ¹H NMR (d₆-DMSO) two isomers, approximately 2:1 δ 8.89 (bd s, 1H), 8.71 (d, J 8.51 Hz; 1H), 8.40 (d, J 8.9 Hz, 1H), 8.14-7.90 (m, 41, 7.56 (d+v bd n, J 14.6 Hz, 2H), 7.01 (bd in, H), 6.65 (dd, J 13.9, 11.5, 1H), 6.27 bd t J 12.4 Hz, 1H), 5.57 (bd m, 1H), 5.12 (t J 5.2 Hz; 1H), 4.90 (bd ‘s’, 2H), 3.90 (bd ‘s’, 2H, 1.58 (bd s, 3H), 1.40 (bd s, 3H). ¹³C NMR (d₆-DMSO) 175.3 (C_{Q}), 153.7 (C_{Q}), 152.5(C_{Q}), 146.9 (CH), 146.2 (CH), 138.5 (C_{Q}), 137.6 (CH), 134.6 (CH), 128.7 (CH), 126.2 (CH), 119.3 (CH), 118.4 (C_{Q}), 113.8 (CH), 105.7 (CH), 92.1 (C_{Q}), 59.3 (CH₂), 58.4 (CH₂), 27.6 (CH₃). λₘₐₓ 735 (DMF) log₁₀ε 4.70; (MeOH) 705; (pyridine) 860.

‘“{4-“{2-‘{3-{2-[N-(2-Hydroxyethyl)quinolin-4(1H)-ylidene]-ethylidene)-2-chloro-1-cyclohexen-1-yl}’-E-ethenyl}”-3-cyano-5,5-dimethyl-2(5H)-furanylidene}”’propanedinitrile (3d) was recovered as a black powder, (13%), m.p. >300° C. ¹H NMR (d₆-DMSO) δ 8.97 (d, J 6.6 Hz, 1H), 8.87 (d, J 8.5 Hz, 1H), 8.47 (d, J 8.9 Hz, 1H), 8.29 (d, J 15.3 Hz, 1H), 8.17 (t, J 7.8, 1H), 7.95 (t, J 7.7 Hz, 1H), 7.61 (d, J 15.3 Hz, 1H) 7.30 (apparent s, 1H), 5.66 (bd, 1H), 5.14 (bd t, —OH), 4.98 (bd t, 2H), 3.90 (m, 2H), 2.79 (m, 2H), 2.59(m, 2H), 1.86 (m, 2H), 1.47 (bd s, 6H). ¹³C NMR (d₆-DMSO) 152.6 (C_{Q}), 147.8 (CH), 140.3 (CH), 138.4 (C_{Q}), 134.8 (CH), 130.0 (C_{Q}), 129.1(CH), 126.8 (CH), 119.4 (CH), 118.9 (CH), 117.5 (C_{Q}), 115.4 (CH), 101.9 (CH), 59.3 (CH₂), 58.8 (CH₂), 27.6 (CH₃), 27.3 (CH₃), 26.2 (CH₂), 21.2 (CH₂). λₘₐₓ 730 (DMF) log₁₀ε 4.72; (MeOH) 710; (pyridine) 865.

### (vi) N-Methylquinolin-2(1H)-ylidene donors—Method A

‘{4{2-[N-Methylquinolin-2(1H)-ylidene]ethenyl}-3-cyano-5,5-dimethyl-2(5H)-furanylidene}’propanedinitrile (7a) was obtained as a dark green, (66%), m.p. >300° C. (Found: MH⁺ m/z 367.15534; C₂₃H₁₈N₄O requires MH⁺ m/z 367.15368 Δ=4.5 ppm). ¹H NMR (d₆-DMSO) two isomers, approximately 2:1, δ 8.70 (bd m, 0.3H), 8.49 (bd m, 0.3H), 8.35 (bd m, 1H), 8.15 (bd m, 1H), 8.04 (dd, J 7.9, 1.2 Hz, 1H), 7.92 (bd m, 1.3H), 7.74 (bd m, 0.3H), 7.65 (bd m, 1H), 6.80 (d, J 13.5 Hz, 0.6H), 6.69 (d, J 12.6 Hz, 0.3H), 6.19 (d, J 13.0 Hz, 0.6H), 5.98 (d, J 11.8 Hz, 0.3H), 41.3 (bd s, 3H), 1.75 (s, 4H), 1.51 (bd s, 2H). Solvent insolubility precluded the recording of meaningful ¹³C NMR data. λₘₐₓ (DMF) 620 log₁₀ε 5.18; (MeOH) 610; (pyridine) 634.

‘{4{4-[N-Methylquinolin-2(1H)-ylidene]-1,3-butadienyl}-3-cyano-5,5-dimethyl-2(5H) furanylidene}’propanedinitrile (7b) was obtained as a dark green microrystalline solid, (66%), m.p. >300° C. (Found: MH³⁰ m/z 393.17099; C₂₅H₂₀N₄O requires MH⁺ m/z 393.17162 Δ=1.6 ppm). ¹H NMR (d₆-DMSO) two isomers, approximately 2:1, δ 8.60-7.65 (5×bd m, 8H), 6.89 (bd m, 1H), 6.51 (bd t, J 12.2 Hz, 1H), 5.78 (bd m, 1H), 4.15 (bd s, 3H), 1.65 (bd s, 4H), 1.47 (bd s, 2H). Solvent insolubility precluded the recording of meaningful ¹³C NMR data. λₘₐₓ (DMF) 710 log₁₀ε5.12; (MeOH) 700; (pyridine) 740.

‘{4{6-[N-Methylquinolin-2(1H)-ylidene]-1,3,5-hexatrienyl}-3-cyano-5,5-dimethyl-2(5H)-furanylidene}’propanedinitrile (7c) was obtained as a emerald green powder, (45%), m.p. 245-248° C. (Found: MH⁺ m/z 419.18664; C₂₇H₂₂N₄O requires MH⁺ m/z 418.18532 Δ=3.1 ppm). Solvent insolubility precluded the recording of meaningful ¹H and ¹³C NMR data. λₘₐₓ 746 (DMF) log₁₀ε 4.85; (MeOH) 700; (pyridine) 850.

### (vii) N-(2-Hydroxyethyl)benzothiazol-2(3H)-ylidene]-donors—Method B

‘{4-{2-[N-(2-hydroxyethyl)benzothiazol-2(3H)-ylidene]-ethenyl}-3-cyano-5,5-dimethyl-2(5H)-furanylidene}’propanedinitrile (4a) was recovered as a grey/green microcrystalline solid, (100%), m.p. >300° C. (Found: MH⁺ m/z 403.12232; C₂₂H₁₈N₄O₂S requires MH⁺ m/z 403.12398 Δ=4.1 ppm). ¹H NMR (d₆-DMSO) δ 8.04 (d, J 5.8 Hz, 1H0, 7.82 (d, J 8.1 Hz, 1H), 7.61 (t, J 7.4 Hz, 1H), 7.48 (t, J 7.4 Hz, 1H), 6.68 (d, J 13.2 Hz, 1H), 6.00 (bd s, 1H), 4.64 (t, J 5.4 Hz, 2H), 4.07 (t, J 5.4 Hz, 2H), 1.62 (s, 6H). Solvent insolubility precluded the recording of meaningful ¹³C NMR data. λₘₐₓ (DMF) 605 log₁₀ε5.18; (MeOH) 598; (pyridine) 614.

‘{4-{4-[N-(2-hydroxyethyl)benzothiazol-2(3H)-ylidene]-1,3-butadienyl}-3-cyano-5,5-dimethyl-2(5H)-furanylidene}’propanedinitrle (4b) was recovered as a grey/green microcrystalline solid, (98%), m.p. 275° C. (Found: MH⁺ m/z 429.13797; C₂₄H₂₀N₄O₂S requires MH⁺ m/z 429.14030 Δ=5.4 ppm). ¹H NMR (d₆-DMSO) δ 8.12 (d, J 7.8 Hz, 1H), 7.91 (d, J 8.3 Hz, 1H), 7.82 (bd m, 2H), 7.64 (t, J 8.2 Hz, 1H), 7.53 (t, J 7.7 Hz, 1H), 6.86 (d, J 13.9 Hz, 1H), 6.41 (‘t’, J 12.4 Hz, 1H), 5.84 (d, J 13.1 Hz, 1H), 5.08 (t, J 5.5 Hz, 1H), 4.61 (nm, 2H), 3.82 (m, 2H), 1.62 (bd s, 6H). ¹³C NMR (d₆-DMSO) 151.5 (CH), 147.4 (CH), 142.2 (C_{Q}), 128.6 (CH), 126.5 (CH), 124.1 (CH), 123.6 (CH), 116.8 (C_{Q}), 115.6 (CH), 107.2 (CH), 106.3 (CH), 94.1 (C_{Q}), 59.0 (CH₂), 50.1 (CH₂), 27.1 (CH₃). λₘₐₓ (DMF) 705 log₁₀ε 5.21; (MeOH) 698; (pyridine) 718.

‘4-{6-[N-(2-hydroxyethyl)benzothiazol-2(3H)-ylidene]-1,3,5-hexatrienyl}-3-cyano-5,5-dimethyl-2(5H)-furanylidene}’propanedinitrile (4c) was recovered as a grey/green microcrystalline solid, (100%), map. >265° C. (Found: M⁺ m/z 454.14768; C₂₆H₂₂N₄O₂S requires M⁺ m/z 454.14580 Δ=4.1 ppm). ¹H NMR (d₆-DMSO) δ 8.21 (d, J 7.8 Hz, 1H), 8.00 (d, J 8.4 Hz, 1H), 7.79 (bd m, c.1H), 7.70 (t, J 7.5 Hz, 1H), 7.61 (t, J 7.5 Hz, 1H), 7.59 (bd m, c.1H), 7.34 (bd m, c.1H), 7.10 (d, J 14.1 Hz, 1H), 6.53 (‘t’, J 12.6 Hz, 1H), 6.34 (bd m, 1H), 5.71 (d, J 12.6 Hz, 1H), 5.18 (bd, 1H), 4.68 (m, 2H), 3.84 (m, 2H), 1.50 (bd s, 6H). Solvent insolubility precluded the recording of meaningful ¹³C NMR data. λₘₐₓ 810 (DMF) log₁₀ε 4.91; (MeOH) 805; (pyridine) 830.

‘{4-{4-[N-(2-hydroxyethyl)benzothiazol-2(3H)-ylidene]-1,3-butadienyl}-5-(4-acetoxy-phenyl)-3-cyano-5-methyl-2(5H)-furanylidene}’propanedinitrie (4d) was recovered as a grey/green microcrystalllne solid, (97%), m.p. 257° C. (Found: MH⁺ m/z 549.16072; C₃₁H₂₄N₄O₄S requires MH⁺ m/z 549.15910 Δ=2.9 ppm). ¹H NMR (d₆-DMSO) δ 8.13 (d, J 7.9 Hz, 1H), 7.94 (d, J 8.2 Hz, 1H), 7.75-7.40 (m, 6H), 7.21 (d, J 8.6 Hz, 2H) 6.90 (bd m, 1H), 6.35 (‘t’, J 12.3 Hz, 1H), 5.91 (bd m, 1H), 5.06 (t, J 5.4 Hz, 1H), 4.60 (m, 2H), 3.81 (nm, 2H), 2.73 (s, 3H), 2.04 (v bd s, 3H). ¹³C NMR (d₆-DMSO) 169.4 (C_{Q}), 151.1 (C_{Q}), 147.0 (C_{Q}), 142.1 (C_{Q}), 128.8 (CH), 127.8 (CH), 126.8 (CH), 124.7 (CH), 123.7 (CH), 122.5 (CH), 116.8 (C_{Q}), 116.0 (CH), 107.5 (CH), 106.5 (CH), 94.8 (C_{Q}), 59.1 (CH₂), 50.3 (CH₂), 25.1 (CH₃), 21.2 (CH₃). λₘₐₓ 705 (DMF) log₁₀ε 5.26.

‘“{4-“{2-‘{3-{2-[N-(2-hydroxyethyl)benzothiazol-2(3H)-ylidene]ethylidene}-2-chloro-1-cyclohexen-1-yl}-E-ethenyl}”-3-cyano-5,5-dimethyl-2(5H)-furanylidene}”’propanedinitrile (4e) was recovered as a dark green powder, (44%), m.p. >300° C. ¹H NMR (d₆-DMSO) δ 8.23 (d, J 7.4 Hz, 1H), 8.13 (d, J 14.7 Hz, 1H), 8.06 (d, J 8.3 Hz, 1H), 7.96 (m, 1H), 7.73 (t, J 8.3 Hz, 1H), 7.63 (t, J 7.4 Hz, 1H), 7.08 (d, J 14.7 Hz, 1H), 5.76 (d, J 13.2 Hz, 1H), 5.11 (t, J 5.9 Hz, 1H, OR), 4.81 (m, 2H), 3.85 (m, 2H), 2.65 (m, 2H), 2.60 (m, 2H), 1.81 (m, 2H), 1.53 (s,6H). Solvent insolubility precluded the recording of meaningful ¹³C NMR data. λₘₐₓ 854 (DMF) log₁₀ε 4.91; (MeOH) 840; (pyridine) 865.

Representative NLO Polyurethane Polymer—35% Chromophore Loading

To a stirred solution of ‘“{4-“{{2-‘{3-{2-[N-(2,3-dihydroxypropyl)quinolin-4(1H)-ylidene]-ethylidene}-2-chlorocyclohexen-1-yl}’-E-ethenyl}”-3-cyano-5,5-dimethyl-2(5H)-furanylidene}”’-propanedinitrile (552 mg, 1.0 mmole) (the N-[2,3-dihydroxypropyl] analogue of 3d) and bisphenol-A (456 mg 2.0 mmole) in anhydrous DMSO was added, in a single portion, toluene-2,4-diisocyanate (TDI) (543 mg 3.0 mmole). The mixture was stirred for 16 hours under a blanket of argon at 80-90° C., after which time the solution was cooled and then filtered through a plug of glass wool into a vigorously stirred volume of methanol (200 ml). Stirring was continued for an additional 60 mins before the suspension was filtered (initially in the absence of a vacuum) through a glass sinter. The resulting intensely green solid polyurethane washed with copious volumes of methanol and then dried before being redissolved in a minimum volume of DMSO and then this solution filtered through a 1.0 micron glass fibre pad. The polyurethane was recovered by precipitation in methanol as described above and isolated as a fine, dark green powder.

### SUMMARY OF OPTOPHORES SYNTHESISED

### 4-Pyridinylidene (4-PY) and 4-quinolinylidene (4-Q) optophores

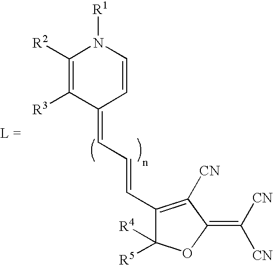

### 4-Pyridinylidene (4-PY)

| | R¹ | R² | R³ | R⁴ | R⁵ | L |
|---|---|---|---|---|---|---|
| 1a | CH₃ | H | H | CH₃ | CH₃ | n = 1 |
| 1b | CH₃ | H | H | CH₃ | CH₃ | n = 2 |
| 1c | CH₃ | H | H | CH₃ | CH₃ | n = 3 |
| 2a | CH₂CH(OH)CH₂OH | H | H | CH₃ | CH₃ | n = 1 |
| 2b | CH₂CH(OH)CH₂OH | H | H | CH₃ | CH₃ | n = 2 |

### 4-Quinolinylidene (4-Q)

| | | | | | |
|---|---|---|---|---|---|
| R¹ | R² | R³ | R⁴ | R⁵ | L |

| | | | | | |
|---|---|---|---|---|---|
| 3a | CH₂CH₂OH | benzo fusion | CH₃ | CH₃ | n = 1 |
| 3b | CH₂CH₂OH | benzo fusion | CH₃ | CH₃ | n = 2 |
| 3c | CH₂CH₂OH | benzo fusion | CH₃ | CH₃ | n = 3 |
| 3d | CH₂CH₂OH | benzo fusion | CH₃ | CH₃ | 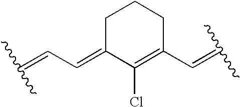 |

### Benzothiazolidinylidene (BT) optophores

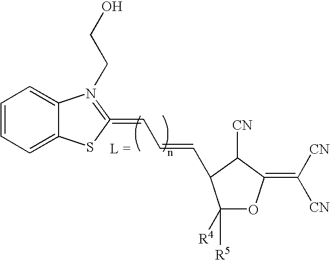

| | R⁴ | R⁵ | L |
|---|---|---|---|
| 4a | CH₃ | CH₃ | n = 1 |
| 4b | CH₃ | CH₃ | n = 2 |
| 4c | CH₃ | CH₃ | n = 3 |
| 4d | CH₃ | p-C₆H₄—OAc | n = 2 |
| 4e | CH₃ | CH₃ | 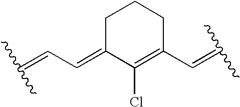 |

### 2-Pyridinylidene (2-PY) and 2-quinolinylidene (2-Q) optophores

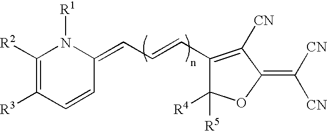

### 2-Pyridinylidene (2-PY)

| | R¹ | R² | R³ | R⁴ | R⁵ | L |
|---|---|---|---|---|---|---|
| 5a | CH₃ | H | H | CH₃ | CH₃ | n = 1 |
| 5b | CH₃ | H | H | CH₃ | CH₃ | n = 2 |
| 5c | CH₃ | H | H | CH₃ | CH₃ | n = 3 |
| 6a | CH₂CH(OH)CH₂OH | H | H | CH₃ | CH₃ | n = 1 |
| 6b | CH₂CH(OH)CH₂OH | H | H | CH₃ | CH₃ | n = 2 |

### 2-Quinolinylidene (2-Q)

| | | | | | |
|---|---|---|---|---|---|
| R¹ | R² | R³ | R⁴ | R⁵ | L |

| | | | | | |
|---|---|---|---|---|---|
| 7a | CH₃ | benzo fusion | CH3 | CH₃ | n = 1 |
| 7b | CH₃ | benzo fusion | CH₃ | CH₃ | n = 2 |
| 7c | CH₃ | benzo fusion | CH₃ | CH₃ | n = 3 |

General Information

¹H- and ¹³C NMR spectra were recorded on a Bruker AVANCE 300 MHz spectrometer and proton multiplicities are defined by the usual notations. The assignments of resonances were made employing DEPT, COSY, HSQC and NOESY pulse sequences.

Uv-vis absorption spectra were recorded on a Hewlett-Packard 8452A diode array spectrophotometer and accurate mass measurements were made on a PE Biosystems Mariner mass spectrometer operating in the electrospray mode. Microanalyses were performed by the Campbell Microanalytical Laboratory, University of Otago, Dunedin, New Zealand and melting points are uncorrected.

Apparatus and experimental procedures for measuring hyper-Raleigh scattering (HRS) have been described previously and are well known in the art β values were determined by using β₈₀₀ for crystal violet chloride (338×10⁻³⁰ esu in methanol) as an external reference. All measurements were performed in DMSO and optical local field correction factors were applied.

Uv-Vis Spectral Characterisation

The electronic absorption spectra of the optophores from all PY, Q and BT series show intense charge-transfer absorption maxima in the 500-850 nm range and reveal the expected red shifts as the extent of conjugation is increased (Table 1). Interestingly, for almost all of the 2- and 4-PY and Q systems which exist as mixtures of rotomers about the π-system, the charge transfer bands are essentially symmetrical in all solvents and show no evidence of an ‘aggregate band’ on the blue side of the absorption. The BT optophores, on the other hand, all exhibit blue-shifted shoulders to their charge transfer bands. Significant windows of transparency exist in the 350-450 nm region of the spectrum for all compounds.

Furthermore, all compounds are negatively solvatochromic; a feature which qualitatively distinguishes these zwitterionic RHS sys from those ‘belonging’ to the LHS. The effect of benzannelation on λₘₐₓ of PY systems is quite pronounced, there being red shifts of up to 150 an in going from PY to Q, for example. The BT series shows not only the effects of benzannelation but also the effects of introducing a component of the π-interconnect which confers a degree of rigidity or configuration locking to the molecule. A compound containing the chlorocyclohexenylidene linkage 4e is red-shifted by approximately 40 nm when compared to its iso-π-electronic series member 4c containing a 7 carbon linker.

TABLE 1
Electronic absorption data for selected chromophores.

| | λₘₐₓ | | λₘₐₓ | λₘₐₓ | |
|---|---|---|---|---|---|
| Com- | (DMF)/ | log₁₀ε | (Methanol)/ | (Pyridine)/ | Δ λₘₐₓ |
| pound | nm | (DMF) | nm | nm | (λ_{pyr}-λMeOH) |
| 1a (4-PY) | 570 | 4.86 | 564 | 600 | 36 |
| 1b (4-PY) | 600 | 4.78 | 592 | 670 | 78 |
| 1c (4-PY) | 615 | 4.76 | 595 | 685 | 90 |
| 2a (4-PY) | 572 | 4.79 | 570 | 598 | 28 |
| 2a (4-PY) | 604 | 4.64 | 598 | 662 | 64 |
| 5a (2-PY) | 556 | 4.90 | 550 | 580 | 30 |
| 5b (2-PY) | 588 | 4.81 | 582 | 662 | 80 |
| 5c (2-PY) | 598 | 4.29 | 580 | 670 | 90 |
| 6a (2-PY) | 556 | 4.72 | 552 | 578 | 26 |
| 6b (2-PY) | 590 | 4.61 | 584 | 652 | 68 |
| 3a (4-Q) | 660 | 5.00 | 654 | 682 | 28 |
| 3b (4-Q) | 735 | 4.88 | 724 | 782 | 58 |
| 3c (4-Q) | 735 | 4.70 | 705 | 860 | 155 |
| 3c (4-Q) | 730 | 4.72 | 710 | 865 | 115 |
| 7a (2-Q) | 620 | 5.18 | 610 | 634 | 24 |
| 7b (2-Q) | 710 | 5.12 | 700 | 740 | 40 |
| 7c (2-Q) | 746 | 4.85 | 700 | 850 | 150 |
| 4a (BT) | 605 | 5.18 | 598 | 614 | 16 |
| 4b (BT) | 705 | 5.21 | 698 | 718 | 20 |
| 4c (BT) | 810 | 4.91 | 805 | 830 | 25 |
| 4e (BT) | 854 | 4.91 | 840 | 865 | 25 |

Hyper Raleigh Scattering Studies

The first-order hyperpolarisabilities, β, of a representative suite of these zwitterionic optophores have been measured using the hyper-Raleigh (HRS) technique with a femtosecond-pulsed fundamental of 800 nm and SH at 400 nm, at which wavelength no two-photon-induced fluorescence was detected by temporal resolution of emitted light signals. The β values are therefore true experimental values with no resonance enhancement. Furthermore, the resonance enhancement due to the fact that the charge-transfer band is in between the fundamental and the second harmonic indicates that the signs for the dynamic β at the measurement wavelength and for the static βₒ are opposite, this being based upon assumptions applied to the two-state model.

HRS-derived β and βₒ values are presented in Table 2 together with calculated values of the ground-state dipole moment μ and βₒ and the corresponding figures of merit (μ.βₒ). In spite of the fact that 800 nm radiation triggered some (not unexpected) photodegradation of various of these optophores, the results clearly follow the expected trend which shows an increasing β as a function of increasing conjugation length. Interestingly, the effects of benzannelation upon the PY systems; that is, in going from 4-PY 1a-c to Q 3a-c, have a marked effect upon the size of βₒ. This is perhaps an indication of the increasing donor strength of Q systems over PY systems. To the extent that βₒ values for the two BT compounds are reliable, then these data again suggest that the BT nucleus is midway in strength between the PY and Q donors.

The figures of merit (μ_{calc}, β_{o(HRS)}) we obtain for the longest of each of the 4-PY and 4-Q optophores (1c and 3c, respectively) (Table 2) are of the same order of magnitude as that reported for the optophores of U.S. Pat. No. 6,067,186, members of which (LHS) class are characterised as having exceptional optical nonlinearities. The highest value of (μ_{calc}, β_{o(HRS)}) reported for an optophore described in U.S. Pat. No. 6,067,186 is 8255×10⁻⁴⁸ esu and those we obtain for the highest members of each of the PY and Q suites (1c and 3c) are 8900×10⁻⁴⁸ and 9384×10⁻⁴⁸ esu, respectively.

TABLE 2
Calculated and experimental values of βₒ and μ, and the corresponding figures
of merit.

| | μ^{a} (calc)/ | βₒ^{a} (calc)/ | βₒ^{b} (found)/ | μ_{(calc)} · β_{o(calc)}/ | μ_{(calc)} · βₒ(found)/ | μ_{(calc).} |
|---|---|---|---|---|---|---|
| Compound | 10⁻¹⁸ esu | 10⁻³⁰ esu | 10⁻³⁰ esu | 10⁻⁴⁸ esu | 10⁻⁴⁸ esu | β_{o(found)/MW} |
| 1a (4-PY) | 17.7 | 148 | 125 | 2620 | 2213 | 7.0 |
| 1b (4-PY) | 17.9 | 268 | 360 | 4749 | 6444 | 18.8 |
| 1c (4-PY) | 17.8 | 343 | 500 | 6105 | 8900 | |
| 5a (2-PY) | 15.6 | 125 | — | 1950 | — | |
| 5b (2-PY) | 15.9 | 240 | — | 3816 | — | |
| 5c (2-PY) | 15.9 | 319 | — | 5072 | — | |
| 3a (4-Q) | 15.5 | 153 | 440 | 2372 | 6820 | 17.2 |
| 3b (4-Q) | 12.7 | 229 | 560 | 2908 | 7112 | 16.8 |
| 3c (4-Q) | 13.8 | 253 | 680 | 3491 | 9384 | 20.9 |
| 7a (2-Q) | 14.7 | 142 | — | 2087 | — | |
| 7b (2-Q) | 14.7 | 247 | — | 3631 | — | |
| 7c (2-Q) | 14.6 | 309 | — | 4511 | — | |
| 4a (BT) | 10.4 | 100 | 240 | 1040 | 2496 | 6.2 |
| 4b (BT) | 12.9 | 173 | 260 | 2232 | 3354 | 7.8 |
| 4c (BT) | 12.8 | 213 | — | 2726 | — | |

^{a}MOPAC/AM1 level using the precise keyword.
^{b}βₒ is the static first hyperpolarisability estimated by using the two-state model. This is derived from β, the dynamic first hyperpolarisability, which was measured using a ferntosecond-pulsed Ti-Sapphire fundamental at 800 nm

The above examples are illustrations of the invention. Those skilled in art will appreciate that numerous adaptions and modifications can be made without departing from the scope and spirit of the invention. Therefore, it is to be understood that the invention may be practiced other than as specifically described herein.

1. A compound of the general Formula I: 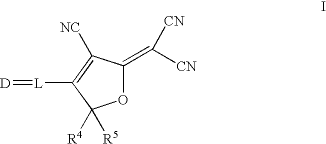
wherein:
D is selected from the group comprising: 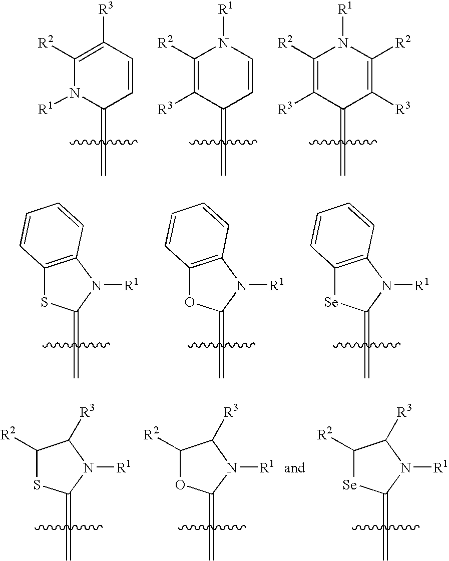
and wherein:
R¹ is alkyl or hydroxyalkyl;
R² and R³ are H, or together with the carbon atoms to which they are attached form a 6-membered aromatic ring;
L is a linking group comprising an optionally substituted chain of 3, 5 or 7 carbon atoms which, together with the double bond linking D to L forms a conjugated polyenic chain; and
R⁴ and R⁵ are independently alkyl, hydroxyalkyl or p-C₆H₄—OAc.

2. A compound of claim 1
wherein:
D is selected from the group comprising: 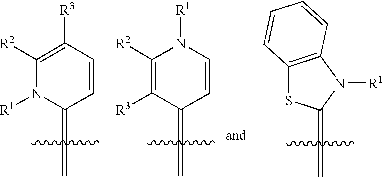
and wherein:
R¹ is alkyl or hydroxyalkyl;
R² and R³ are H, or together with the carbon atoms to which they are attached form a 6-membered aromatic ring;
L is a linking group comprising an optionally substituted chain of 3, 5 or 7 carbon atoms which, together with the double bond linking D to L forms a conjugated polyenic chain; and
R⁴ and R⁵ are independently alkyl, hydroxyalkyl or p-C₆H₄—OAc.

3. A compound of claim 1 wherein L is an optionally substituted chain of 3 or 5 carbon atoms which, together with the double bond linking D to L forms a conjugated polyenic chain.

4. A compound of claim 3 wherein R¹ is dihydroxyalkyl.

5. A compound of claim 1 wherein R² and R³ together with the carbon atoms to which they are attached form a 6-membered aromatic ring.

6. A compound of claim 1 wherein R⁴ and R⁵ are independently alkyl or hydroxyalkyl.

7. A compound of claim 1, represented by 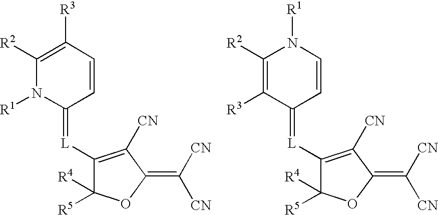
wherein:
R¹ is CH₃, CH₂CH₂OH, CH₂CH(OH)CH₂OH or alkyl chain of up to 30 carbon atoms;
R² and R³ are H, or together with the carbon atoms to which they are attached form a 6-membered aromatic ring;
one of R⁴ or R⁵ is hydroxyalkyl; and
L is an optionally substituted chain of 5 carbon atoms which, together with the double bond linking D to L forms a conjugated polyenic chain.

8. A compound of claim 7 wherein R¹ is dihydroxyalkyl.

9. A compound selected from the group comprising:
[4{2-(N-Methylpyridin-4(1H)-ylidene)ethenyl}-3-cyano-5,5-dimethyl-2(5H)furanylidene}]propanedinitrile;
[4{4-(N-Methylpyridin-4(1H)-ylidene)-1,3-butadienyl}-3-cyano-5,5-dimethyl-2(5H)-furanylidene]propanedinitrile;
[4{6-(N-Methylpyridin-4(1H)-ylidene)-1,3,5-hexatrienyl}-3-cyano-5,5-dimethyl-2(5H)-furanylidene]propanedinitrile;
‘{4{2-[N-(2,3-Dihydroxypropyl)pyridine-4(1H)-ylidene]ethenyl}-3-cyano-5,5-dimethyl-2(5H)-furanylidene}’propanedinitrile;
‘{4{4-[(2,3-Dihydroxypropyl)pyridin-4(1H)-ylidene]-1,3-butadienyl}-3-cyano-5,5-dimethyl-2(5H)-furanylidene}’propanedinitrile;
[4{2-(N-Methylpyridin-2(1H)-ylidene)ethenyl}-3-cyano-5,5-dimethyl-2(5H)-furanylidene]propanedinitrile;
[4{4-(N-Methylpyridin-2(1H)-ylidene]-1,3-butadienyl}-3-cyano-5,5-dimethyl-2(5H)-furanylidene]propanedinitrile;
[4{6-(N-Methylpyridin-2(1H)-ylidene)-1,3,5-hexatrienyl}-3-cyano-5,5-dimethyl-2(5H)-furanylidene]propanedinitrile;
‘{4{2-[N-(2,3-Dihydroxypropyl)pyridin-2(1H)-ylidene]ethenyl}-3-cyano-5,5-dimethyl-2(5H)-furanylidene}’propanedinitrile;
‘{4{4-[N-(2,3-Dihydroxypropyl)pyridin-2(1H)-ylidene]-1,3,5-butadienyl}-3-cyano-5,5-dimethyl-2(5H)-furanylidene}’propanedinitrile;
‘{4{2-[N-(2-Hydroxyethyl)quinolin-4(1H)-ylidene]ethenyl}-3-cyano-5,5-dimethyl-2(5H)-furanylidene}’propanedinitrile;
‘{4{4-[N-(2-Hydroxyethyl)quinolin-4(1H)-ylidene]-1,3-butadienyl}-3-cyano-5,5-dimethyl-2(5H)-furanylidene}’propanedinitrile;
‘{4{6-[N-(2-Hydroxyethyl)quinolin-4(1H)-ylidene]-1,3,5-hexatrienyl}-3-cyano-5,5-dimethyl-2(5H)-furanylidene}’propanedinitrile;
‘“{4-”{2-‘{3-{2-[N-(2-Hydroxyethyl)quinolin-4(1H)-ylidene]-ethylidene}-2-chloro-1-cyclohexen-1-yl}’-E-ethenyl}“-3-cyano-5,5-dimethyl-2(5H)-furanylidene}”’propanedinitrile;
‘{4{2-[N-Methylquinolin-2(1H)-ylidene]ethenyl}-3-cyano-5,5-dimethyl-2(5H)-furanylidene}’propanedinitrile;
‘{4{4-[N-Methylquinolin-2(1H)-ylidene]-1,3-butadienyl}-3-cyano-5,5-dimethyl-2(5H)-furanylidene}’propanedinitrile;
‘{4{6-[N-Methylquinolin-2(1H)-ylidene]-1,3,5-hexatrienyl}-3-cyano-5,5-dimethyl-2(5H)-furanylidene}’propanedinitrile;
‘{4-{2-[N-(2-hydroxyethyl)benzothiazol-2(3H)-ylidene]-ethenyl}-3-cyano-5,5-dimethyl-2(5H)-furanylidene}’propanedinitrile;
‘{4-{4-[N-(2-hydroxyethyl)benzothiazol-2(3H)-ylidene]-1,3-butadienyl}-3-cyano-5,5-dimethyl-2(5H)-furanylidene}’propanedinitrile;
‘{4-{6-[N-(2-hydroxyethyl)benzothiazol-2(3H)-ylidene]-1,3,5-hexatrienyl}-3-cyano-5,5-dimethyl-2(5H)-furanylidene}’propanedinitrile;
‘{4-{4-[N-(2-hydroxyethyl)benzothiazol-2(3H)-ylidene]-1,3-butadienyl}-5-(4-acetoxy-phenyl)-3-cyano-5-methyl-2(5H)-furanylidene}’propanedinitrile; and
‘“{4-”{2-‘{3-{2-[N-(2-hydroxyethyl)benzothiazol-2(3H)-ylidene]ethylidene}-2-chloro-1-cyclohexen-1-yl}’-E-ethenyl}“-3-cyano-5,5-dimethyl-2(5H)-furanylidene}”’propanedinitrile.

10. A method of preparing a compound of Formula I as defined in claim 1 comprising:
(a) reacting a compound of Formula II: 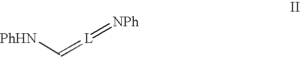
wherein L is defined in claim 1, with a compound of Formula III: 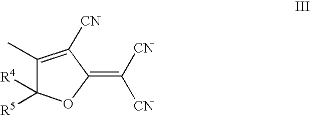
wherein R⁴ and R⁵ are as defined in claim 1, to form a compound of Formula IV: 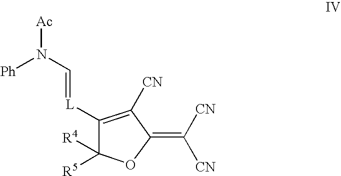
(b) reacting the compound of Formula IV from step (a) with a donor compound to form a compound of Formula I, wherein the donor compound bears a donor group selected from the group comprising:

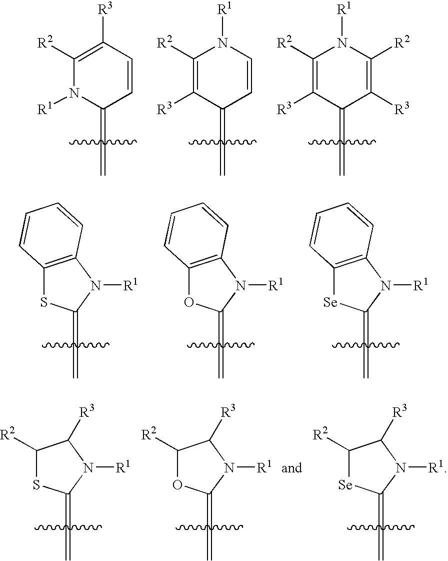

11. A method of claim 10 comprising:
(a) reacting a compound of Formula II: 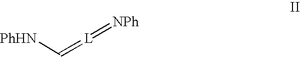
wherein L is defined in claim 1, with a compound of Formula III: 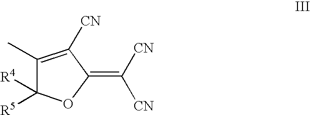
wherein R⁴ and R⁵ are as defined in claim 1, to form a compound of Formula IV: 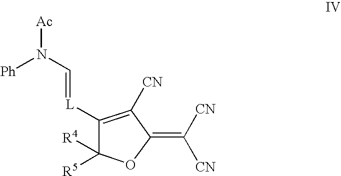
(b) reacting the compound of Formula IV from step (a) with an azinium or azolium donor derivative of Formula V, VI, or VII, where X is halogen and R¹, R², R³ are defined in claim 1, to form a compound of Formula I, wherein:

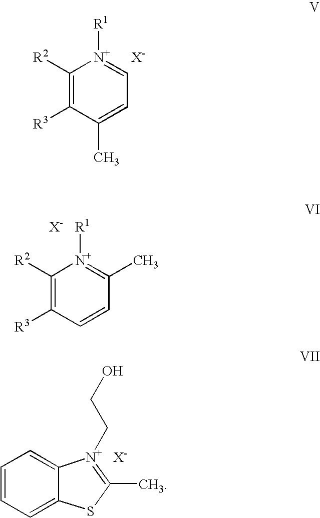

12. A composite material prepared from a polymerisation mixture comprising:
(a) a compound of formula I of claim 1 or a derivative thereof; and
(b) at least a further polymerisable material.

13. A composite material of claim 12 comprising a modified polyurethane, polycarbonate, polyamic acid polyimide, or a mixture thereof, which includes substituents derived from a compound of formula I.

14. An optoelectronic device comprising the composite material of claim 12.

15. A method of data transmission comprising transmitting light through a composite material of claim 12.

16. A compound of claim 2 wherein L is an optionally substituted chain of 3 or 5 carbon atoms which, together with the double bond linking D to L forms a conjugated polyenic chain.

17. A compound of claim 16 wherein R¹ is dihydroxyalkyl.
